(19) United States
(12) Patent Application Publication
(10) Pub. No.: US 2018/0078768 A1
(43) Pub. Date: Mar. 22, 2018

(54) METHOD AND APPARATUS FOR PAIN MANAGEMENT USING HEART SOUNDS

(71) Applicant: Boston Scientific Neuromodulation Corporation, Valencia, CA (US)

(72) Inventors: Pramodsingh Hirasingh Thakur, Woodbury, MN (US); Jianwen Gu, Valecia, CA (US); David J. Ternes, Roseville, MN (US); David L. Perschbacher, Coon Rapids, MN (US); Bryan Allen Clark, Forest Lake, MN (US); James John Kleinedler, Plumouth, MN (US)

(21) Appl. No.: 15/687,925

(22) Filed: Aug. 28, 2017

Related U.S. Application Data
(60) Provisional application No. 62/395,641, filed on Sep. 16, 2016.

(51) Int. Cl. A61N 1/36; A61B 7/04; A61B 5/00; A61B 5/042; A61B 5/08

(52) U.S. Cl. CPC A61N 1/36071; A61B 7/04; A61B 5/4824; A61B 5/0816; A61N 1/36135; A61B 5/042; A61N 1/36062

(57) ABSTRACT

An example of a pain management system may include a heart sound sensor, a heart sound sensing circuit, a heart sound detector, a parameter generator, and a pain analyzer. The heart sound sensor may be configured to sense a heart sound signal. The heart sound sensing circuit may be configured to process the heart sound signal. The heart sound detector may be configured to detect heart sounds using the processed heart sound signal. The parameter generator may be configured to generate parameter(s) using the detected heart sounds. The pain analyzer may be configured to analyze the parameter(s) for a quantitative indication of pain, and include a signal metric generator that may be configured to generate a signal metric using the one or more parameters and a pain score generator that may be configured to generate a pain score indicative of a degree of pain using the signal metric.

### CLAIM OF PRIORITY

This application claims the benefit of priority under 35 U.S.C. § 119(e) of U.S. Provisional Patent Application Ser. No. 62/395,641, filed on Sep. 16, 2016, which is herein incorporated by reference in its entirety.

### CROSS REFERENCE TO RELATED APPLICATIONS

This application is related to commonly assigned U.S. Provisional Patent Application Ser. No. 62/400,313, entitled “SYSTEMS AND METHODS FOR CLOSED-LOOP PAIN MANAGEMENT”, filed on Sep. 27, 2016 and U.S. Provisional Patent Application Ser. No. 62/400,336, entitled “METHOD AND APPARATUS FOR PAIN MANAGEMENT USING OBJECTIVE PAIN MEASURE”, filed on Sep. 27, 2016, which are incorporated by reference in their entirety.

### TECHNICAL FIELD

This document relates generally to medical devices and more particularly to a pain management system that produces a measure of pain using heart sounds.

### BACKGROUND

Pain may result from an injury, a disease (e.g., arthritis, fibromyalgia), or even a medical treatment (e.g., certain cancer treatment). Various treatments are applied for pain management, such as medication, psychotherapy, electrical stimulation, thermal therapy, and their various combinations. Examples of electrical stimulation for pain management include Transcutaneous Electrical Nerve Stimulation (TENS) delivered by a TENS unit and Spinal Cord Stimulation (SCS) that may be delivered by an implantable neuromodulation systems. Pain treatment may be prescribed based on an assessment of a patient's symptoms and underlying conditioning and titrated based on the patient's response to the treatment. As pain is not directly measurable by a machine, the assessment of the condition and the titration of the therapy may depend on questioning the patient.

### SUMMARY

An example (e.g., “Example 1”) of a system for providing a patient with pain management may include a heart sound sensor, a heart sound sensing circuit, a heart sound detector, a parameter generator, and a pain analyzer. The heart sound sensor may be configured to sense a heart sound signal. The heart sound sensing circuit may be configured to process the heart sound signal. The heart sound detector may be configured to detect heart sounds using the processed heart sound signal. The parameter generator may be configured to generate one or more parameters using the detected heart sounds. The pain analyzer may be configured to analyze the one or more parameters for a quantitative indication of pain, and include a signal metric generator and a pain score generator. The signal metric generator may be configured to generate a signal metric using the one or more parameters. The pain score generator may be configured to generate a pain score indicative of a degree of pain using the signal metric.

In Example 2, the subject matter of Example 1 may optionally be configured to further include a pain relief device configured to deliver a pain relief therapy and a control circuit configured to control the delivery of the pain relief therapy using the pain score.

In Example 3, the subject matter of Example 2 may optionally be configured to further include a posture sensor configured to sense a posture of the patient, and configured such that the control circuit is configured to control the delivery of the pain relief therapy using the pain score, one or more thresholds, and the posture of the patient.

In Example 4, the subject matter of any one or any combination of Examples 2 and 3 may optionally be configured such that the pain relief device includes an implantable neuromodulator configured to deliver spinal cord stimulation (SCS).

In Example 5, the subject matter of any one or any combination of Examples 1 to 4 may optionally be configured such that the pain score generator is configured to generate the pain score by trending the signal metric.

In Example 6, the subject matter of Example 5 may optionally be configured such that the pain score generator is configured to trend a specified percentile of the signal metric when the patient is at a specified activity level.

In Example 7, the subject matter of any one or any combination of Examples 5 and 6 may optionally be configured such that the pain score generator is configured to trend the signal metric for different postures of the patient.

In Example 8, the subject matter of any one or any combination of Examples 1 to 7 may optionally be configured such that the heart sound detector is configured to detect first heart sounds (S1), and the parameter generator is configured to generate an S1 amplitude being an amplitude of the detected S1.

In Example 9, the subject matter of any one or any combination of Examples 1 to 8 may optionally be configured such that the heart sound detector is configured to detect second heart sounds (S2), and the parameter generator is configured to generate an S2 amplitude being an amplitude of the detected S2.

In Example 10, the subject matter of any one or any combination of Examples 1 to 9 may optionally be configured to further include cardiac sensing electrodes configured to sense one or more cardiac signals, a cardiac sensing circuit configured to process the sensed one or more cardiac signals, and an electrical event detector configured to detect one or more cardiac electrical events using the processed one or more cardiac signals, and configured such that the parameter generator is configured to generate the one or more parameters using the detected heart sounds and the detected cardiac electrical events.

In Example 11, the subject matter of Example 10 may optionally be configured such that the parameter generator is configured to generate one or more cardiac intervals each measured between a detected heart sound of the detected heart sounds and a detected cardiac event of the detected cardiac electrical events.

In Example 12, the subject matter of any one or any combination of Examples 10 and 11 may optionally be configured such that the parameter generator is configured to generate one or more cardiac contractility parameters each indicative of cardiac contractility measured from the one or more cardiac signals using the detected cardiac electrical events.

In Example 13, the subject matter of any one or any combination of Examples 1 to 12 may optionally be configured to further include a respiratory sensor configured to sense a respiratory signal, a respiratory sensing circuit configured to process the sensed respiratory signal, and a respiratory parameter detector configured to detect one or more respiratory parameters using the processed sensed respiratory signal, and configured such that the parameter generator is configured to generate the one or more parameters using the detected heart sounds and the one or more respiratory parameters.

In Example 14, the subject matter of Example 13 may optionally be configured such that the heart sound detector is configured to detect first heart sounds (S1), and the parameter generator is configured to generate an S1 modulation parameter indicative of respiratory modulation of S1 amplitude using the detected heart sounds and the one or more respiratory parameters.

In Example 15, the subject matter of any one or any combination of Examples 13 and 14 may optionally be configured such that the heart sound detector is configured to detect second heart sounds (S2), and the parameter generator is configured to generate an S2 modulation parameter indicative of respiratory modulation of S2 amplitude using the detected heart sounds and the one or more respiratory parameters.

An example (e.g., “Example 16”) of a method for providing a patient with pain management is also provided. The method may include receiving a heart sound signal, detecting heart sounds using the received heart sound signal, generating one or more parameters using the detected heart sounds, generating a signal metric using the one or more parameters, generating a pain score indicative of a degree of pain using the signal metric, delivering a pain relief therapy, and controlling the delivery of the pain relief therapy automatically using the pain score.

In Example 17, the subject matter of delivering the pain relief therapy as found in Example 16 may optionally include delivering spinal cord stimulation (SCS).

In Example 18, the subject matter of generating the pain score as found in any one or any combination of Examples 16 and 17 may optionally include trending the signal metric.

In Example 19, the subject matter of trending the signal metric as found in Example 18 may optionally include trending a specified percentile of the signal metric for different postures of the patient when the patient is at a specified activity level.

In Example 20, the subject matter of detecting the heart sounds as found in any one or any combination of Examples 16 to 19 may optionally include detecting at least one of first heart sounds (S1) or second heart sounds (S2), and the subject matter of generating the one or more parameters as found in Example 18 may optionally include generating at least one of an S1 amplitude being an amplitude of the detected S1 or an S2 amplitude being an amplitude of the detected S2.

In Example 21, the subject matter of Example 20 may optionally further include receiving one or more cardiac signals, detecting one or more cardiac electrical events using the receiving one or more cardiac signals, and generating the one or more parameters using the detected heart sounds and the detected cardiac electrical events.

In Example 22, the subject matter of generating the one or more parameters as found in Example 21 may optionally include generating one or more cardiac intervals each measured between a detected heart sound of the detected heart sounds and a detected cardiac event of the detected cardiac electrical events.

In Example 23, the subject matter of generating the one or more parameters as found in Example 21 may optionally include generating one or more cardiac contractility parameters each indicative of cardiac contractility measured from the one or more cardiac signals using the detected cardiac electrical events.

In Example 24, the subject matter of any one or any combination of Examples 20 to 23 may optionally further include receiving a respiratory signal, detecting one or more respiratory parameters using the processed sensed respiratory signal, and generating the one or more parameters using the detected heart sounds and the one or more respiratory parameters.

In Example 25, the subject matter of detecting the heart sounds as found in Example 24 may optionally include detecting at least one of first heart sounds (S1) or second heart sounds (S2), and the subject matter of generating the one or more parameters as found in Example 34 may optionally include generating one or more of a first heart sound (S1) modulation parameter indicative of respiratory modulation of S1 amplitude or a second heart sound (S2) modulation parameter indicative of respiratory modulation of S2 amplitude using the detected heart sounds and the one or more respiratory parameters.

This Summary is an overview of some of the teachings of the present application and not intended to be an exclusive or exhaustive treatment of the present subject matter. Further details about the present subject matter are found in the detailed description and appended claims. Other aspects of the disclosure will be apparent to persons skilled in the art upon reading and understanding the following detailed description and viewing the drawings that form a part thereof, each of which are not to be taken in a limiting sense. The scope of the present disclosure is defined by the appended claims and their legal equivalents.

### BRIEF DESCRIPTION OF THE DRAWINGS

The drawings illustrate generally, by way of example, various embodiments discussed in the present document. The drawings are for illustrative purposes only and may not be to scale.

FIG. 1 illustrates an embodiment of a pain management system.

FIG. 2 illustrates an embodiment of a pain monitoring circuit, such as may be used in the pain management system of FIG. 1.

FIG. 3 illustrates another embodiment of the pain monitoring circuit, such as may be used in the pain management system of FIG. 1.

FIG. 4 illustrates an implantable neuromodulation system, such as one in which the pain management system of FIG. 1 may be implemented, and portions of an environment in which the implantable neuromodulation system may be used.

FIG. 5 illustrates an embodiment of a method for pain management.

FIG. 6 illustrates another embodiment of the method for pain management.

FIG. 7 illustrates an embodiment of a method for trending a signal metric quantitatively indicating pain.

### DETAILED DESCRIPTION

In the following detailed description, reference is made to the accompanying drawings which form a part hereof, and in which is shown by way of illustration specific embodiments in which the invention may be practiced. These embodiments are described in sufficient detail to enable those skilled in the art to practice the invention, and it is to be understood that the embodiments may be combined, or that other embodiments may be utilized and that structural, logical and electrical changes may be made without departing from the spirit and scope of the present invention. References to “an”, “one”, or “various” embodiments in this disclosure are not necessarily to the same embodiment, and such references contemplate more than one embodiment. The following detailed description provides examples, and the scope of the present invention is defined by the appended claims and their legal equivalents.

This document discusses a method and system for indicating pain that can be used in a closed-loop pain management system. While different patient may have different sensitivity and tolerance to pain, optimization of a pain relief therapy may be difficult and/or inefficiency when being dependent on patent questioning and manual programming. The present system provides for an objective and quantitative measure of pain that can be used in an automated closed-loop pain management system for treating pain and/or symptoms associated with pain, such as a spinal cord stimulation (SCS) system, to optimize pain relief. Examples of other applications of pain management with an objective and quantitative measure of pain can include diagnostic procedures and optimization of therapy settings as performed in a clinic or hospital, in-hospital monitoring such as for opioid dosing during surgery, and ambulatory monitoring such as for recommending administration of pharmaceuticals or for assessing efficacy of trial therapeutic interventions.

One of the hallmarks of pain is an increase in sympathetic tone, which reflects on hemodynamic signals such as heart sounds. In various embodiments, the present system can sense heart sounds, extract one or more features from the heart sounds, and analyze the extracted one or more features to quantify pain. In various embodiments, the outcome of the analysis, such as one or more scores quantifying a degree of pain, can be used to control a pain management therapy, such as being used as an input in an automated closed-loop SCS or other pain relieving electrical stimulation system.

In this document, a “heart sound signal” includes any signal indicative of heart sounds. “Heart sounds” include audible mechanical vibrations caused by cardiac activity that can be sensed with a microphone and audible and inaudible mechanical vibrations caused by cardiac activity that can be sensed with an accelerometer or optical sensor. Heart sounds include the “first heart sound” or S1, the “second heart sound” or S2, the “third heart sound” or S3, the “fourth heart sound” or S4, and their various sub-components. S1 is known to be indicative of, among other things, mitral valve closure, tricuspid valve closure, and aortic valve opening. S2 is known to be indicative of, among other things, aortic valve closure and pulmonary valve closure. S3 is known to be a ventricular diastolic filling sound often indicative of certain pathological conditions including heart failure. S4 is known to be a ventricular diastolic filling sound resulted from atrial contraction and is usually indicative of pathological conditions. The term “heart sound” hereinafter refers to any heart sound (e.g., S1) and any components thereof (e.g., M1 component of S1, indicative of Mitral valve closure). Unless noted otherwise, S1, S2, S3, and S4 refer to the first, second, third, and fourth heart sounds, respectively, as a heart sound type, or as one or more occurrences of the corresponding type heart sounds, depending on the context.

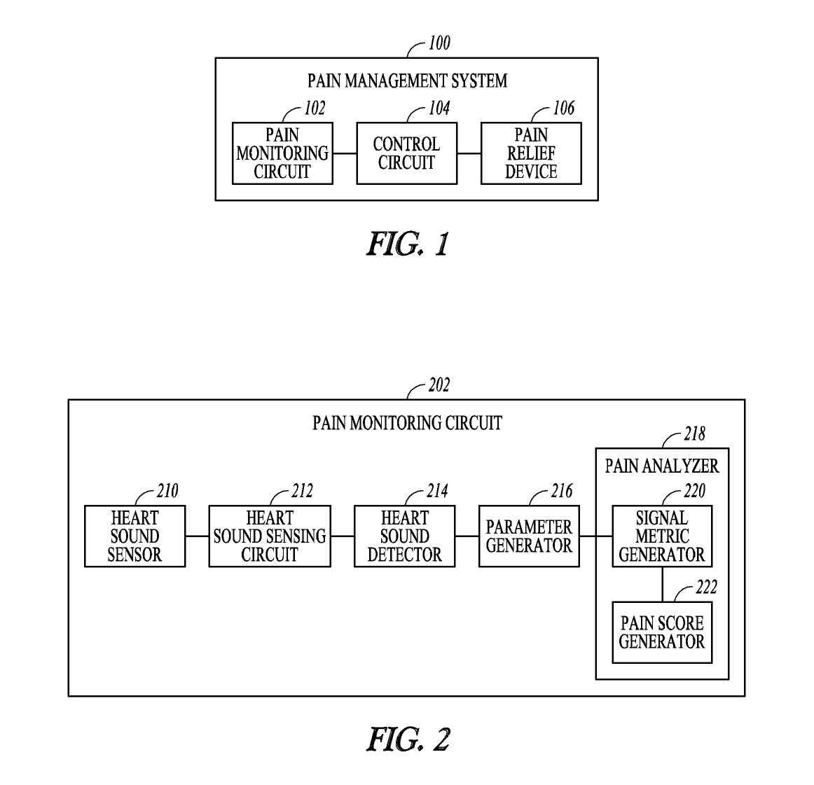

FIG. 1 illustrates an embodiment of a pain management system 100. System 100 includes a pain monitoring circuit 102, a control circuit 104, and a pain relief device 106. In various embodiments, system 100 provides a patient with closed loop pain management in which delivery of one or more pain relief therapies can be controlled automatically using signals sensed from the patient.

Pain monitoring circuit 102 can sense one or more physiological signals and produce the one or more pain indicating signals using the one or more physiological signals. Pain relief device 106 can deliver one or more pain relief therapies. Examples of such one or more pain relief therapies can include any one or any combination of spinal cord stimulation (SCS), dorsal root ganglia (DRG) stimulation, deep brain stimulation (DBS), motor cortex stimulation (MCS), transcranial direct current stimulation (tDCS), transcutaneous spinal direct current stimulation (tsDCS), trigeminal nerve stimulation, occipital nerve stimulation, vagus nerve stimulation (VNS), sacral nerve stimulation, pudendal nerve stimulation, sphenopalatine ganglion stimulation, sympathetic nerve modulation, multifidus muscle stimulation, adrenal gland modulation, carotid baroreceptor stimulation, transcutaneous electrical nerve stimulation (TENS), transcranial magnetic stimulation (TMS), tibial nerve stimulation, transcranial magnetic stimulation (TMS), radiofrequency ablation (RFA), pulsed radiofrequency ablation, ultrasound therapy, high-intensity focused ultrasound (HIFU), optical stimulation, optogenetic therapy, magnetic stimulation, other peripheral tissue stimulation therapies, other peripheral tissue denervation therapies, drug therapy (such as delivered from a drug pump), and nerve blocks or injections (such as pharmaceuticals or biologics). Control circuit 104 can analyze the one or more physiological signals and control the delivery of the one or more pain relief therapies using an outcome of the analysis. For example, the outcome of the analysis can include a pain score indicative of a degree (intensity) of pain, and control circuit 104 controls the delivery of the one or more pain relief therapies using the pain score and one or more thresholds. Other factors affecting the degree of pain and/or effectiveness of the one or more pain relief therapies can also be included in the analysis. For example, the patient's posture can be monitored, and control circuit 104 controls the delivery of the one or more pain relief therapies using the pain score, the one or more thresholds, and the patient's posture.

In various embodiments, system 100 can be an implantable system placed inside the patient, an external system, a percutaneous system, a system with a combination of any two or more of implantable components, external components, and/or percutaneous system. Examples of pain relief device 106 can include, but are not limited to, a neuromodulator (e.g., an external neuromodulator to deliver TENS, or an implantable neuromodulator to deliver electrical stimulation such as SCS or peripheral nerve stimulation), a drug delivery device to deliver one or more pain suppression agents, and a light emitter to deliver a light therapy (e.g., low level laser therapy or infrared therapy).

In various embodiments, system 100 can control the delivery of the one or more pain relief therapies from pain relief device 106 automatically using feedback control with the one or more physiological signals sensed by pain monitoring circuit 102 as input. In various embodiments, the one or more physiological signals include at least a heart sound signal. Control circuit 104 detects one or more parameters from the heart sound signal and derive an objective measure of the patient's pain based on the one or more parameters. For example, increased amplitude of S1 (resulting from increase cardiac contractility due to increased sympathetic drive) and/or increased amplitude of S2 when the patient is at rest can be an objective quantitative measure of the patient's pain. In various embodiments, the one or more physiological signals include at least a heart sound signal and one or more additional signals needed for a heart-sound based analysis. Control circuit 104 detects one or more parameters from the heart sound signal and the one or more additional signals, and derive an objective measure of the patient's pain based on the one or more parameters. For example, decreased pre-ejection period (PEP) with decreased raw left ventricular ejection time (LVET) but increased heart rate-corrected LVET can be an objective quantitative measure of the patient's pain. PEP can be measured using the heart sound signal and a cardiac signal indicative of the patient's cardiac electrical events. Modulation of S1 amplitude and/or S2 amplitude by respiration can be an objective quantitative measure of the patient's pain. The S1 amplitude variability and/or S2 amplitude variability are expected to be lower with chronic pain (resulting from reduced respiratory modulation due to increased sympathetic drive). Heart sound amplitude variability can be measured as an explicit modulation response to respiration, or can be measured independent of a respiration signal by observing the variability in the heart sounds over a period of time that the patient is known to be at a specified activity level (e.g., at rest or within a specified range, as determined using an accelerometer). The variability can be graphically depicted by plotting the ΔS1 between two successive beat versus S1 at the current beat for all the beats observed over a given period. A low-intensity pain will result in a good modulation of S1 resulting in a greater area (which can be referred to as a “footprint”), whereas an increase in pain intensity will reduce the S1 modulations and thus the area of the footprint. A similar footprint can also be created using S2 amplitude or cardiac intervals. A trend of the area of the footprint can be used to track the changes in pain intensities over time. In various embodiments, such one or more parameters can be used in various combinations to derive an objective quantitative measure of the patient's pain. In some embodiments, such one or more parameters, including their various combinations, can further be combined with one or more parameters measured from the cardiac signal and indicative of the patient's cardiac contractility (e.g., amplitude of R-wave) to derive an objective quantitative measure of the patient's pain.

In various embodiments, circuits of system 100, including various embodiments of its components discussed in this document, may be implemented using a combination of hardware and software. For example, pain monitoring circuit 102, including its various embodiments discussed in this document, and control circuit 104 may be implemented using an application-specific circuit constructed to perform one or more particular functions or a general-purpose circuit programmed to perform such function(s). Such a general-purpose circuit includes, but is not limited to, a microprocessor or a portion thereof, a microcontroller or portions thereof, and a programmable logic circuit or a portion thereof.

FIG. 2 illustrates an embodiment of a pain monitoring circuit 202, which represent an example of pain monitoring circuit 102. Pain monitoring circuit 202 includes a heart sound sensor 210, a heart sound sensing circuit 212, a heart sound detector 214, a parameter generator 216, and a pain analyzer 218.

Heart sound sensor 210 senses a heart sound signal. In various embodiments, heart sound sensor 210 can include an accelerometer or a microphone. Heart sound sensing circuit 212 processes the heart sound signal. The processing can include removal of unwanted signal components, such as patient's physical activity sensed by the accelerometer or background noise sensed by the microphone. Heart sound detector 214 detects heart sounds using the processed heart sound signal. In various embodiment, the heart sounds detected for deriving an objective quantitative measure of the patient's pain includes S1 and S2. Parameter generator 216 generates one or more parameters using the detected heart sounds. Pain analyzer 218 analyzes the one or more parameters for a quantitative indication of pain. In the illustrated embodiment, pain analyzer 218 includes a signal metric generator 220 and a pain score generator 222. Signal metric generator 220 generates a signal metric using the one or more parameters. Pain score generator 222 generates a pain score indicative of a degree of pain using the signal metric. In various embodiments, pain analyzer 218 can be configured to apply any analysis for producing a quantitative indication of pain using the one or more parameters.

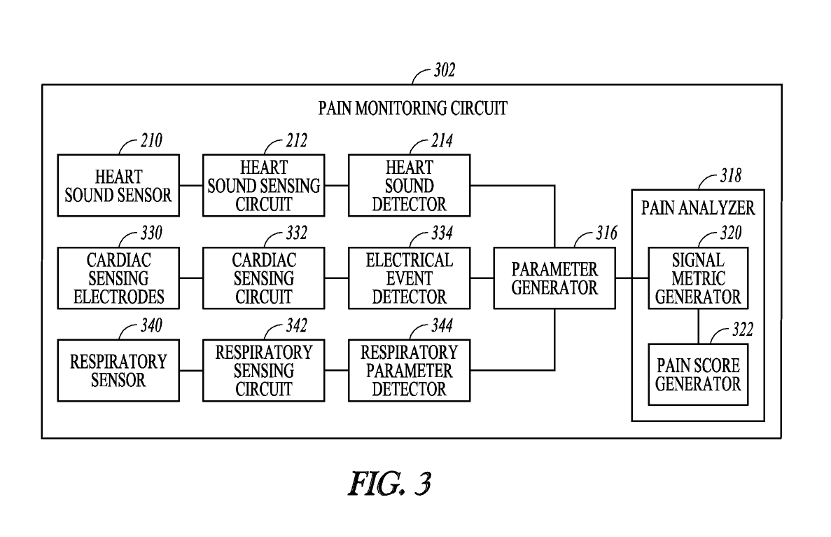

FIG. 3 illustrates an embodiment of a pain monitoring circuit 302, which represent another example of pain monitoring circuit 102. Pain monitoring circuit 302 can perform functions of pain monitoring circuit 202 with additional circuitry to sense cardiac and/or respiratory signals such that the one or more parameters generated for pain analysis can further include one or more heart sound related parameters that can be measured using the heart sound signal and the cardiac and/or respiratory signals.

In the illustrated embodiment, pain monitoring circuit 302 include heart sound sensor 210, heart sound sensing circuit 212, heart sound detector 214, cardiac sensing electrodes 330, a cardiac sensing circuit 332, an electrical event detector 334, a respiratory sensor 340, a respiratory sensing circuit 342, a respiratory parameter detector 344, a parameter generator 316, and a pain analyzer 318. In various embodiments, the cardiac sensing system (cardiac sensing electrodes 330, cardiac sensing circuit 332, and electrical event detector 334) and respiratory sensing system (respiratory sensor 340, respiratory sensing circuit 342, and respiratory parameter detector 344) may be optional. Pain monitoring circuit 202 can include the heart sound sensing system (heart sound sensor 210, heart sound sensing circuit 212, and heart sound detector 214) only, or the heart sound sensing system plus either one or both of the cardiac sensing system and the respiratory sensing system, depending on which one or more parameters are used for the pain analysis, as further discussed in this document.

Cardiac sensing electrodes 330 are used to sense one or more cardiac signals. Cardiac sensing circuit 332 processes the sensed one or more cardiac signals. Electrical event detector 334 detects one or more cardiac electrical events using the processed one or more cardiac signals. In various embodiments, the one or more cardiac signals can include surface electrocardiogram (ECG), wireless ECG (including subcutaneous ECG), and/or intracardiac electrogram. The one or more cardiac electrical events can include P-wave, Q-wave, R-wave, S-wave, and/or T-wave, depending on which one or more parameters are used for the pain analysis, as further discussed in this document. “Surface ECG” includes a cardiac electrical signal sensed with electrodes attached onto the exterior surface of the skin. “Wireless ECG” includes a signal approximating the surface ECG, acquired without using surface (non-implantable, skin contact) electrodes. “Subcutaneous ECG” is a form of wireless ECG and includes a cardiac electrical signal sensed through electrodes implanted in subcutaneous tissue, such as through electrodes incorporated onto an implantable medical device that is subcutaneously implanted. As reflected in their corresponding morphologies, the surface ECG results from electrical activities of the entire heart. The wireless ECG, including but not being limited to the subcutaneous ECG, has a morphology that approximates that of the surface ECG and reflects electrical activities of a substantial portion of the heart, up to the entire heart. Examples for sensing wireless ECG signals including subcutaneous ECG signals is discussed in U.S. Pat. No. 7,299,086, entitled “WIRELESS ECG IN IMPLANTABLE DEVICES”, assigned to Cardiac Pacemakers, Inc., which is incorporated herein by reference in its entirety. One or more wireless ECG signals may be available, for example, when the patient is using an implantable pacemaker, implantable cardioverter defibrillator, or an implantable cardiac monitoring device. “Intracardiac electrogram” includes a cardiac electrical signal sensed with at least one electrode placed in or on the heart. One or more intracardiac electrographic signals may be available, for example, when the patient is using an implantable pacemaker or implantable cardioverter defibrillator. In one embodiment, cardiac sensing circuit 212 removes unwanted components of the sensed one or more cardiac signals, such as pacing artifacts when the patient uses a pacemaker.

Respiratory sensor 340 senses a respiratory signal. Respiratory sensing circuit 342 process the sensed respiratory signal. Respiratory parameter detector 344 detects one or more respiratory parameters using the processed sensed respiratory signal. The respiratory signal is a physiologic signal indicative of respiratory cycles and various other respiratory parameters. In one embodiment, respiratory sensor 340 includes an impedance sensor that senses a transthoracic impedance signal indicative of respiration. In another embodiment, the respiratory sensor includes an implantable pulmonary artery pressure (PAP) sensor or a portion thereof. An example of the implantable PAP sensor is discussed in U.S. Pat. No. 7,566,308, entitled “METHOD AND APPARATUS FOR PULMONARY ARTERY PRESSURE SIGNAL ISOLATION”, assigned to Cardiac Pacemakers, Inc., which is incorporated by reference herein in its entirety. In one embodiment, the respiratory sensor includes an external sensor that senses the expansion and contraction of the chest or a portion thereof. The processed respiratory signal (produced by respiratory sensing circuit 342) is indicative of respiratory cycles and can allow for detection of one or more respiratory parameters such as respiratory cycle length, inspiration period, expiration period, non-breathing period, tidal volume, and minute ventilation. In one embodiment, respiratory sensing circuit 342 removes unwanted components of the sense respiratory signal to isolate the respiratory components of the physiologic signal. One example includes isolating the respiratory components of a PAP signal, which is discussed in U.S. Pat. No. 7,566,308. The one or more of the respiratory parameters detected by respiratory parameter detector 344 include any one or more parameters detectable from the processed respiratory signal and needed for the pain analysis, as further discussed in this document.

Parameter generator 316 generates the one or more parameters using the one or more physiological signals. In various embodiments, the one or more parameters can include, but are not limited to, any one or any combination of (1)-(4) below.
(1) One or more heart sound amplitudes, each measured as a peak amplitude of the heart sound signal during a detected heart sound, or a root-mean-square (RMS) value of the measured peak amplitude, a total area between the amplitude curve and a baseline, or a parameter measured from an envelope fitted to the heart sound morphology (demodulated amplitude), such as an amplitude of the envelope at a certain point or an area under a portion of the envelope. Examples include S1 amplitude and S2 amplitude. In various embodiments, one of the following (a-c) can be generated by parameter generator 316:
   a) S1 amplitude;
   b) S2 amplitude; and
   c) S1 amplitude and S2 amplitude.
(2) One or more cardiac intervals, each measured between a cardiac electrical event and a heart sound. Examples include: (i) PEP, measured as the time interval between a Q or R-wave and the subsequently adjacent S1; (ii) LVET, measured as the time interval between the S1 and the subsequently adjacent S2; (iii) Systolic Interval (SI), measured as the time interval between a Q or R-wave and the subsequently adjacent S2; and (iv) Diastolic interval (DI), measured as the time interval between S2 and the subsequently adjacent Q or R-wave). In various embodiments, one of the following (a-o) can be generated by parameter generator 316:
   a) PEP;
   b) LVET;
   c) SI;
   d) DI;
   e) PEP and LVET;
   f) PEP and SI;
   g) PEP and DI;
   h) LVET and SI;
   i) LVET and DI;
   j) SI and DI;
   k) PEP, LVET, and SI;
   l) PEP, LVET, and DI;
   m) PEP, SI, and DI;
   n) LVET, SI, and DI;
   o) PEP, LVET, SI, and DI.
(3) One or more heart sound modulation parameters, each indicative of respiratory modulation of a heart sound amplitude. Examples include S1 modulation parameter indicative of respiratory modulation of S1 amplitude and S2 modulation parameter indicative of respiratory modulation of S2 amplitude. An example of a heart sound modulation parameter includes a heart sound amplitude variability being breath-to-breath variance of a heart sound amplitude such as the S1 amplitude or S2 amplitude. In various embodiments, one of the following (a-c) can be generated by parameter generator 316:
   a. S1 modulation parameter;
   b. S2 modulation parameter; and
   c. S1 modulation parameter and S2 modulation parameter.
(4) One or more cardiac contractility parameters; each indicative of cardiac contractility measured from the one or more cardiac signals. An example of a cardiac contractility parameter includes an R-wave amplitude. In various embodiments, such one or more cardiac contractility parameters can be generated by parameter generator 316.

Pain analyzer 318 analyzes the one or more parameters generated by parameter generator 316 for a quantitative indication of pain. In the illustrated embodiment, pain analyzer 318 includes a signal metric generator 320 and a pain score generator 322. Signal metric generator 316 generates a signal metric using the one or more parameters. Pain score generator 322 generates a pain score indicative of a degree of pain using the signal metric. In various embodiments, pain score generator 322 can generate the pain score by trending the signal metric. In various embodiments, pain score generator 322 can trend a low percentile of the signal metric at night when the patient is a specified activity level (e.g., at rest or within a specified range, as determined using an accelerometer), trend a low percentile of the signal metric during daytime when the patient is a specified activity level (e.g., at rest or within a specified range, as determined using an accelerometer), and or trend the low percentile (e.g., approximately 5^{th} or 10^{th} percentile) of the signal metric for different postures. This percentile number can be determined to ensure that the lower tail of the distribution is included, while not being locked into spurious outliers. In various embodiments, pain score generator 322 can also be configured to generate the pain score using the signal metric as discussed in this document and one or more other signal metrics such as discussed in U.S. Provisional Patent Application Ser. No. 62/400,313, entitled “MULTI-SENSOR ALGORITHMS FOR CLOSED-LOOP PAIN MANAGEMENT”, filed on ______ (Attorney Docket No. 6279.222PRV) and U.S. Provisional Patent Application Ser. No. 62/400,336, entitled “METHOD AND APPARATUS FOR PAIN MANAGEMENT USING OBJECTIVE PAIN MEASURE”, filed on Sep. 27, 2016, assigned to Cardiac Pacemakers, Inc., which are incorporated by reference herein in their entirety. The one or more other signal metrics may be needed when, for example, the heart-sound based signal metric as discussed in this document is considered insufficient by itself as an objective quantitative measure of pain that can be used to control delivery of a pain relief therapy.

Examples of the one or more parameters generated by parameter generator 316 and used by signal metric generator 320 to generate the signal metric can include, but are not limited to, one of the following (A-O):
A. The one or more heart sound amplitudes (1);
B. The one or more cardiac intervals (2);
C. The one or more heart sound modulation parameters (3);
D. The one or more cardiac contractility parameters (4);
E. (1) and (2);
F. (1) and (3);
G. (1) and (4);
H. (2) and (3);
I. (2) and (4);
J. (3) and (4);
K. (1), (2), and (3);
L. (1), (2), and (4);
M. (1), (3), and (4);
N. (2), (3), and (4); and
O. (1), (2), (3), and (4).

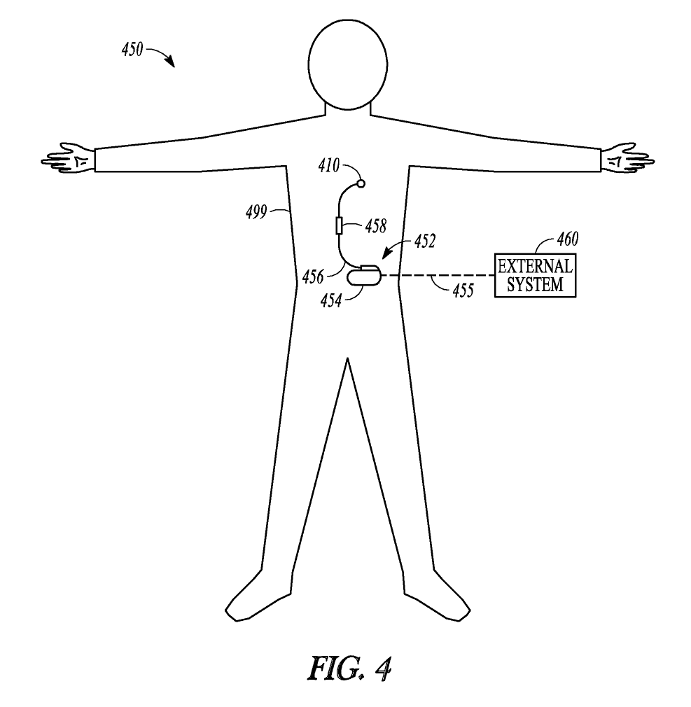

FIG. 4 illustrates an implantable neuromodulation system 450 and portions of an environment in which system 450 may be used. System 450 includes an implantable system 452, an external system 460, and a telemetry link 455 providing for wireless communication between implantable system 452 and external system 460. Implantable system 452 is illustrated in FIG. 4 as being implanted in the patient's body 499.

Implantable system 452 can include an implantable neuromodulator (also referred to as an implantable pulse generator, or IPG) 454, a lead system 456, and electrodes 458 and a heart sound sensor 410 incorporated onto lead system 456. In various embodiments, additional one or more electrodes can be incorporated onto implantable neuromodulator 454. In the illustrated embodiment, heart sound sensor 410, which represents an embodiment of heart sound sensor 210, is incorporated into lead system 452 and to be positioned in or near the thoracic region. In another embodiment, heart sound sensor 410 can be embedded in implantable neuromodulator 454, which can be placed in the lumbar region. In still another embodiment, heart sound sensor 410 can be a separate device, such as an implantable device, that can communicate with implantable neuromodulator 454 wirelessly via telemetry. In various embodiments, system 450 can also include cardiac electrodes, a respiratory sensor, and/or a posture sensor when such one or more sensors are needed for generating parameters need for the signal metric. Such sensors can each be incorporated into lead system 452, incorporated onto or into implantable neuromodulator 454, or communicate with implantable neuromodulator 454 wirelessly via telemetry.

External system 460 can include one or more external (non-implantable) devices each allowing the user and/or the patient to communicate with implantable system 452. In some embodiments, external system 460 includes a programming device intended for a user such as a physician or other caregiver to initialize and adjust settings for implantable neuromodulator 454 and a remote control device intended for use by the patient. For example, the remote control device may allow the patient to turn implantable neuromodulator 454 on and off and/or adjust certain patient-programmable parameters controlling delivery of a neuromodulation therapy.

In various embodiments, implantable neuromodulator 454 can deliver a pain relief neuromodulation therapy such as a SCS or PNS therapy. Pain management system 100, including the various embodiments of its elements discussed in this document, can be implemented in system 450. In various embodiments, system 100, including the various embodiments of its elements discussed in this document, can be implemented entirely in implantable neuromodulator 454 only, or implemented in both implantable neuromodulator 454 and external system 460.

The sizes and shapes of the elements of implantable system 452 and their location in body 499 are illustrated by way of example and not by way of restriction. System 450 is discussed as a specific application of pain management according to various embodiments of the present subject matter. In various embodiments, the present subject matter may be applied in any type of pain management in controlling delivery of one or more pain relief energy and/or agents.

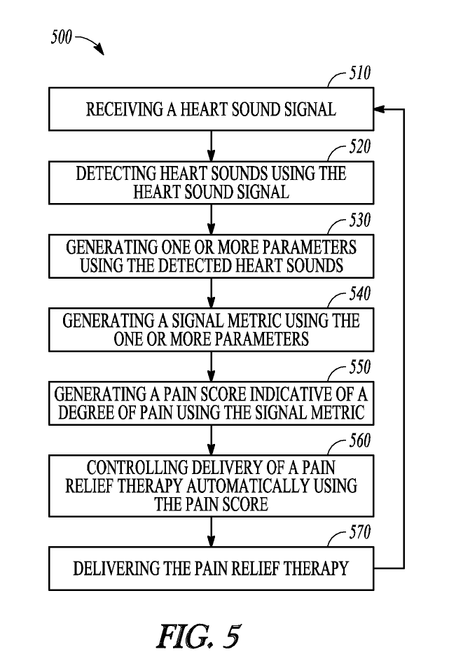

FIG. 5 illustrates an embodiment of a method 500 for pain management with automatic feedback control. In one embodiment, system 100 is configured to perform method 500, and pain monitoring circuit 202 is configured to perform at least steps 510, 520, 530, 540, and 550.

At 510, a heart sound signal is received. In various embodiments, the heard sound can be received from a heart sound sensor such as an accelerometer or a microphone. At 520, heart sounds are detected using the heart sound signal. At 530, one or more parameters are generated using the detected heart sounds. At 540, a signal metric is generated using the one or more parameters. Types of the heart sound detected at 520 and types of the one or more parameters generated at 530 depend on the signal metric, with examples including S1 amplitude and S2 amplitude. At 550, a pain score indicative of a degree of pain is generated using the signal metric. At 560, delivery of a pain relief therapy is controlled automatically using the pain score. At 570, the pain relief therapy is delivered. Method 500 is continuously performed to start, stop, and adjust the delivery of the pain therapy based on the pain score.

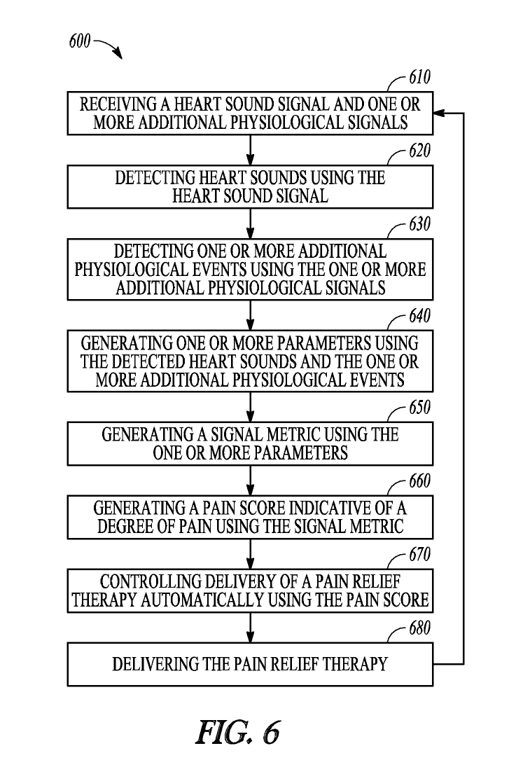

FIG. 6 illustrates another embodiment of the method 600 for pain management. In one embodiment, system 100 is configured to perform method 600, and pain monitoring circuit 302 is configured to perform at least steps 610, 620, 630, 640, 650, and 660.

At 610, a heart sound signal and one or more additional physiological signals are received. In various embodiments, the heard sound can be received from a heart sound sensor such as an accelerometer or a microphone. The one or more additional physiological signals can include one or more cardiac signals and/or a respiratory signal. At 620, heart sounds are detected using the heart sound signal. At 630, one or more additional physiological events are detected using the one or more additional physiological signals. At 640, one or more parameters are generated using the detected heart sounds and the detected one or more additional physiological events. At 650, a signal metric is generated using the one or more parameters. Types of the heart sound detected at 620, types of the one or more additional physiological events detected at 630, and types of the one or more parameters generated at 640 depend on the signal metric, with examples given in Table 1. At 660, a pain score indicative of a degree of pain is generated using the signal metric. At 670, delivery of a pain relief therapy is controlled automatically using the pain score. At 680, the pain relief therapy is delivered. Method 600 is continuously performed to start, stop, and adjust the delivery of the pain therapy based on the pain score.

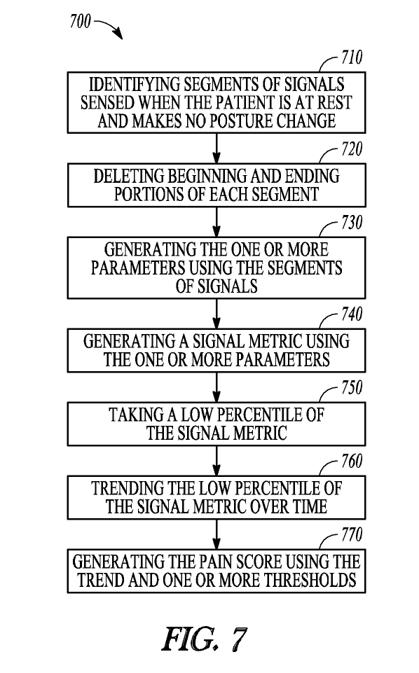

FIG. 7 illustrates an embodiment of a method 700 for trending a signal metric quantitatively indicating pain. Method 700 can be performed by pain score generator as part of method 500 or by pain score generator as part of method 600.

At 710, segments of signals sensed when the patient is a specified activity level (e.g., at rest or within a specified range, as determined using an accelerometer) and makes no posture change is identified. The patient being at the specified activity level without posture changes helps reduce the confounding impact of increased contractility due to activities of daily living. Examples of the signals include the heart sound signal as sensed by pain monitoring circuit 202, or the heart sound signal plus one or both of the cardiac and respiratory signals as sensed by pain monitoring circuit 302. In one embodiment, segments of signals sensed at night (e.g., 12 midnight to 6 am) when the patient is at rest (such as confirmed using an accelerometer) and makes no posture change (such as confirmed using a posture sensor) is identified. In another embodiment, segments of signals sensed during daytime (or anytime) when the patient is a specified activity level (e.g., at rest or within a specified range, as determined using an accelerometer) and makes no posture change (such as confirmed using a posture sensor) is identified. In one embodiment, segment of signals sensed immediately following a transition from a specified non-zero activity level (not at rest) to zero (at rest) is used as a surrogate for the heart sounds sensed at the specified activity level, to overcome the difficulty in sensing activity level and heart sounds simultaneously when activity signals overwhelm heart sounds. This is a reasonable approximation because physiology does not change substantially within the first few seconds following the transition. In various embodiments, method 700 is repeated for various postures of the patient. A trend that increases in one posture and does not increase in a different posture may indicate posture dependent sub-optimal pain relief therapy and hence a need for separate therapy parameters for different postures of the patient.

At 720, the beginning and ending portions (e.g., about 5 minutes) of each identified segment are deleted to ensure quality of signals for the pain analysis. At 730, one or more parameters (such as the one or more parameters generated at 530 or 640) are generated using the identified segments of signals. At 740, a signal metric (such as the signal metric generated at 540 or 650) is generated using the one or more parameters. At 750, a low percentile (such as approximately 5^{th} percentile or 10^{th} percentile) of the signal metric is taken. This percentile number can be determined to ensure that the lower tail of the distribution is included, while not being locked into spurious outliers. At 760, the low percentile of the signal metric is trended over time. In one embodiment, the low percentile of the signal metric is trended with one value a day while the method 500 or 600 is performed for the patient. At 770, the pain score (such as the pain score generated at 550 or 660) using the trend and one or more thresholds.

It is to be understood that the above detailed description is intended to be illustrative, and not restrictive. Other embodiments will be apparent to those of skill in the art upon reading and understanding the above description. The scope of the invention should, therefore, be determined with reference to the appended claims, along with the full scope of equivalents to which such claims are entitled.

What is claimed is:

1. A system for providing a patient with pain management, the system comprising:
a heart sound sensor configured to sense a heart sound signal;
a heart sound sensing circuit configured to process the heart sound signal;
a heart sound detector configured to detect heart sounds using the processed heart sound signal;
a parameter generator configured to generate one or more parameters using the detected heart sounds; and
a pain analyzer configured to analyze the one or more parameters for a quantitative indication of pain, the pain analyzer including:
a signal metric generator configured to generate a signal metric using the one or more parameters; and
a pain score generator configured to generate a pain score indicative of a degree of pain using the signal metric.

2. The system of claim 1, further comprising:
a pain relief device configured to deliver a pain relief therapy; and
a control circuit configured to control the delivery of the pain relief therapy using the pain score.

3. The system of claim 2, wherein the pain relief device comprises an implantable neuromodulator configured to deliver spinal cord stimulation (SCS).

4. The system of claim 3, wherein the pain score generator is configured to generate the pain score by trending the signal metric.

5. The system of claim 4, wherein the heart sound detector is configured to detect at least one of first heart sounds (S1) or second heart sounds (S2), and the parameter generator is configured to generate at least one of an S1 amplitude being an amplitude of the detected S1 or an S2 amplitude being an amplitude of the detected S2.

6. The system of claim 4, further comprising:
cardiac sensing electrodes configured to sense one or more cardiac signals;
a cardiac sensing circuit configured to process the sensed one or more cardiac signals; and
an electrical event detector configured to detect one or more cardiac electrical events using the processed one or more cardiac signals,
and wherein the parameter generator is configured to generate the one or more parameters using the detected heart sounds and the detected cardiac electrical events.

7. The system of claim 6, wherein the parameter generator is configured to generate one or more cardiac intervals each measured between a detected heart sound of the detected heart sounds and a detected cardiac event of the detected cardiac electrical events.

8. The system of claim 6, wherein the parameter generator is configured to generate one or more cardiac contractility parameters each indicative of cardiac contractility measured from the one or more cardiac signals using the detected cardiac electrical events.

9. The system of claim 4, further comprising:
a respiratory sensor configured to sense a respiratory signal;
a respiratory sensing circuit configured to process the sensed respiratory signal; and
a respiratory parameter detector configured to detect one or more respiratory parameters using the processed sensed respiratory signal,
and wherein the parameter generator is configured to generate the one or more parameters using the detected heart sounds and the one or more respiratory parameters.

10. The system of claim 4, wherein the heart sound detector is configured to detect at least one of first heart sounds (S1) or second heart sounds (S2), and the parameter generator is configured to generate one or more of a first heart sound (S1) modulation parameter indicative of respiratory modulation of S1 amplitude or a second heart sound (S2) modulation parameter indicative of respiratory modulation of S2 amplitude using the detected heart sounds and the one or more respiratory parameters.

11. A method for providing a patient with pain management, the method comprising:
receiving a heart sound signal;
detecting heart sounds using the received heart sound signal;
generating one or more parameters using the detected heart sounds;
generating a signal metric using the one or more parameters;
generating a pain score indicative of a degree of pain using the signal metric;
delivering a pain relief therapy; and
controlling the delivery of the pain relief therapy automatically using the pain score.

12. The method of claim 11, wherein delivering the pain relief therapy comprises delivering spinal cord stimulation (SCS).

13. The method of claim 11, wherein generating the pain score comprises trending the signal metric.

14. The method of claim 13, wherein trending the signal metric comprises trending a specified percentile of the signal metric for different postures of the patient when the patient is at a specified activity level.

15. The method of claim 13, wherein detecting the heart sounds comprises detecting at least one of first heart sounds (S1) or second heart sounds (S2), and generating the one or more parameters comprises generating at least one of an S1 amplitude being an amplitude of the detected S1 or an S2 amplitude being an amplitude of the detected S2.

16. The method of claim 15, further comprising:
receiving one or more cardiac signals;
detecting one or more cardiac electrical events using the receiving one or more cardiac signals; and
generating the one or more parameters using the detected heart sounds and the detected cardiac electrical events.

17. The method of claim 16, wherein generating the one or more parameters comprises generating one or more cardiac intervals each measured between a detected heart sound of the detected heart sounds and a detected cardiac event of the detected cardiac electrical events.

18. The method of claim 16, wherein generating the one or more parameters comprises generating one or more cardiac contractility parameters each indicative of cardiac contractility measured from the one or more cardiac signals using the detected cardiac electrical events.

19. The method of claim 18, further comprising:
receiving a respiratory signal;
detecting one or more respiratory parameters using the processed sensed respiratory signal; and
generating the one or more parameters using the detected heart sounds and the one or more respiratory parameters.

20. The method of claim 19, wherein detecting the heart sounds comprises detecting at least one of first heart sounds (S1) or second heart sounds (S2), and generating the one or more parameters comprises generating one or more of a first heart sound (S1) modulation parameter indicative of respiratory modulation of S1 amplitude or a second heart sound (S2) modulation parameter indicative of respiratory modulation of S2 amplitude using the detected heart sounds and the one or more respiratory parameters.
